(19) United States
(12) Patent Application Publication
(10) Pub. No.: US 2006/0289007 A1
(43) Pub. Date: Dec. 28, 2006

(54) Breath actuated nasal drug delivery system

(76) Inventors: Robert Williams, Raleigh, NC (US); Thomas Ruckdeschel, Apex, NC (US); Matthew Khare, Willow Springs, NC (US); Dan Deaton, Apex, NC (US)

Correspondence Address: KOS PHARMACEUTICALS, INC., 1 CEDAR BROOK DRIVE, CRANBURY, NJ, 08512-3618, US

(21) Appl. No.: 11/418,527

(22) Filed: May 3, 2006

Related U.S. Application Data
(63) Continuation-in-part of application No. 11/160,493, filed on Jun. 27, 2005.

(51) Int. Cl. A61M 15/00

(52) U.S. Cl. 128/203.15

(57) ABSTRACT

A nasal drug delivery system and method of delivering a predetermined amount of medication to the nasal epithelia. The system including a container containing a substance to be delivered into the nasal passages of a user. The nasal drug delivery system also includes a nasal shaft extending from the drug delivery system for placement in or around the nose of user, the nasal shaft is in fluid communication with a valve of the container, and an oral shaft, extending from the drug delivery system for placement in the mouth of a user. The nasal drug delivery system also comprises a pressure activated diaphragm trigger, wherein upon application of pressure to the oral shaft triggers the release of a predetermined amount of a substance from the container to the nasal shaft. The method includes steps of inserting the oral shaft into the mouth of a user, inserting the nasal shaft into or around the nose of a user, pressurizing the oral shaft which activates the trigger, and releasing a predetermined amount of medicament from the container into the nasal shaft and into the nasal passage of the user.

This application is a continuation-in-part of U.S. Patent Application No. 11,160,493, filed Jun. 27, 2005, pending.

### FIELD OF THE INVENTION

The present invention relates to an drug delivery device for delivery of liquid and powdered drugs to the nasal cavity. More particularly, the present invention relates to a pressure activated metered-dose inhaler for delivery of drug substances via the nose and nasal passages.

### BACKGROUND OF THE INVENTION

It is well known that the nasal epithelia are effective organs for absorption of drugs into the body. Because of absorption via the nasal epithelia bypasses the Blood Brain Barrier, it is one of the most effective methods of transmitting systemic pharmaceutical products. Currently used applicators to the nasal epithelia are syringed nose drops, pump spray devices, and fluorinated propellant metered dose inhalers or MDI's.

It has been observed that when administering drugs via the nose and the nasal epithelia particle or droplet size has significant impact on absorption. Smaller droplets have been shown to impact on the higher nasal turbinates which promotes better absorption into the body.

FDA Guidelines require testing to demonstrate that only minimal amounts of drug from a nasal delivery device be deposited beyond the nasal passageway and find its way into the pulmonary region. This presents problems for propellant type approaches, which although they typically produce the smallest droplet size, the pressure of the propellant can easily cause the drug to escape the nasal passageways and thus be deposited for example in the lungs.

The present invention is directed at overcoming these problems associated with the prior art drug delivery systems and provide a means of delivering a desired amount of drug to the nasal epithelium while preventing entry into the pulmonary tract and the lungs.

### SUMMARY OF THE INVENTION

It is an objective of the present invention to provide an apparatus and method for application of medication to the nasal passages of a user.

It is a further objective of the present invention to provide a nasal drug delivery system that has the ability to provide extremely small particles needed to coat the superior turbinates and epithelia yet prevent unwanted aerosolized droplets from entering the pulmonary system. In one embodiment the present invention is a nasal drug delivery system including a container containing a substance to be delivered into the nasal passages of a user. The nasal drug delivery system also includes a nasal shaft, extending from the drug delivery system for placement in or around the nose of user, the nasal shaft is in fluid communication with a valve of the container, and an oral shaft, extending from the drug delivery system for placement in the mouth of a user. The nasal drug delivery system also comprises a pressure activated diaphragm trigger means, wherein upon application of pressure to the oral shaft the trigger releases a predetermined amount of a substance from the container to the nasal shaft. In another embodiment, the present invention comprises a method of delivering a predetermined amount of medication to the nasal epithelia of a user including the steps of providing a drug applicator having a container containing a substance to be delivered, a nasal shaft, extending from the drug applicator for placement in or around the nose of user, said nasal shaft in fluid communication with a valve of said container, an oral shaft, extending from the drug applicator for placement in the mouth of a user, and a pressure activated diaphragm trigger means. The method also includes steps of inserting the oral shaft into the mouth of a user, inserting the nasal shaft into or around the nose of a user, pressurizing the oral shaft which activates the trigger, and releasing a predetermined amount of medicament from the container into the nasal shaft and into the nasal passages of the user.

The various features of novelty which characterize the invention are pointed out in particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

The following detailed description, given by way of example and not intended to limit the present invention solely thereto, will best be appreciated in conjunction with the accompanying drawings, wherein like reference numerals denote like elements and parts, in which:

FIG. 1 depicts a nasal drug delivery system according to one aspect of the present invention in a closed position;

FIG. 2 depicts a nasal drug delivery system according to one aspect of the present invention in an open position;

FIG. 3 depicts a nasal drug delivery system according to one aspect of the present invention having a person blowing into the mouthpiece;

FIG. 4 depicts a nasal drug delivery system according to one aspect of the present invention in a following release of the triggering mechanism;

FIG. 5 depicts a nasal drug delivery system according to one aspect of the present invention in a closed position;

FIG. 6 depicts a side view of a drug discharge nozzle according to one aspect of the present invention;

FIGS. 6A-C depict cross-sectional views of the drug discharge nozzle shown in FIG. 6 cut along lines A, B, and C, respectively;

FIG. 7 depicts a nasal drug delivery system according to one aspect of the present invention showing a user's blowing action and the release of the drug into the nose shaft;

FIG. 8 depicts a close-up view of a cam as shown in FIG. 1; and

FIG. 9 depicts a close-up view of a follower as shown in FIG. 1.

### DETAILED DESCRIPTION

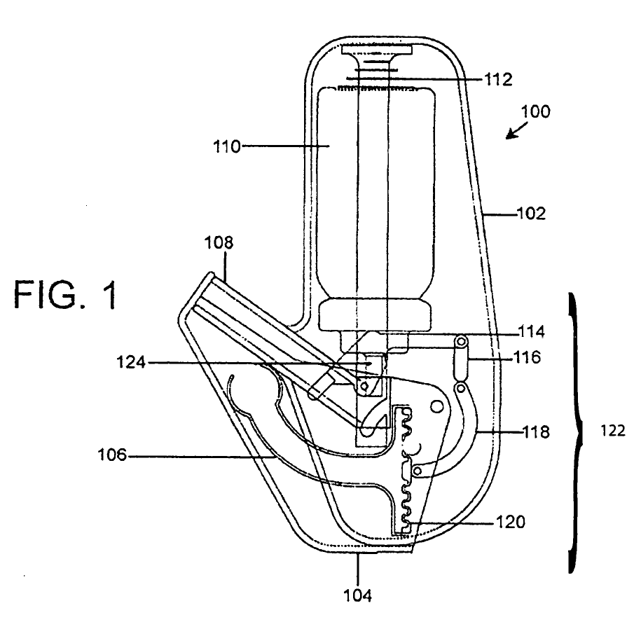

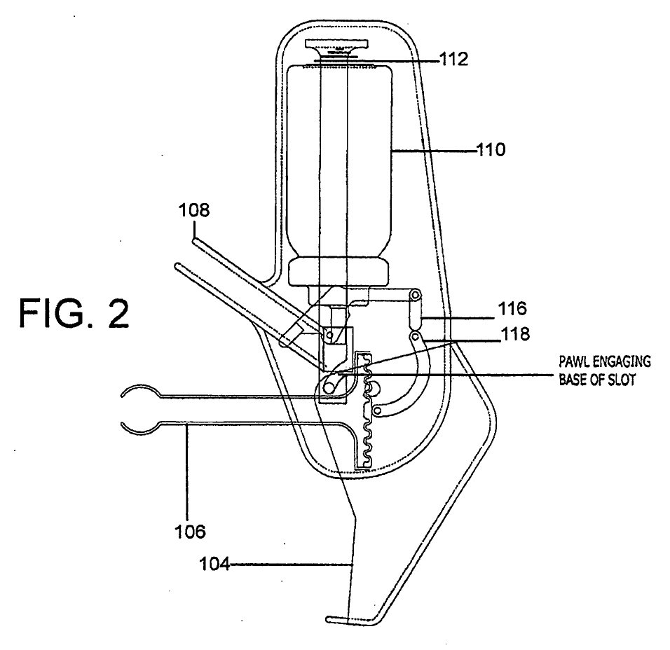

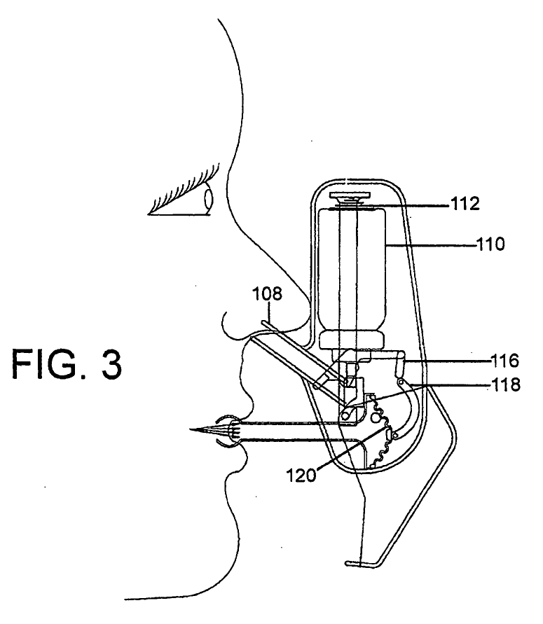

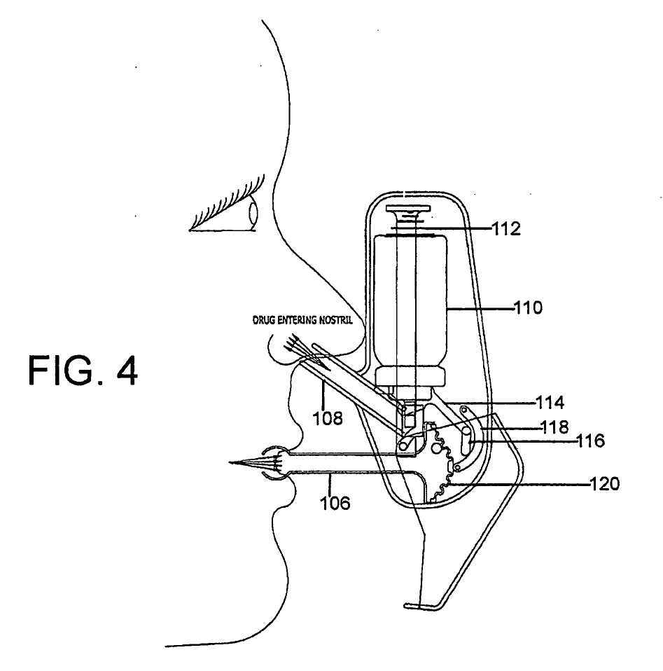

FIG. 1 shows a nasal drug delivery system 100 according to one aspect of the present invention. The nasal drug delivery system 100 includes a housing 102, having a mouthpiece cover 104. The mouthpiece cover 104 protects a mouthpiece 106, the mouthpiece 106 may be formed of a flexible material as shown in FIGS. 1 and 2. The mouthpiece is in fluid communication with a diaphragm 120, which is located in the housing 102. The mouthpiece 106 may also be in fluid communication with a nosepiece 108, as will be discussed below. The housing 102 also contains a drug storage container 110, such as a pressurized MDI. The nasal delivery system 100 also contains a follower assembly 114 having a follower 116 coupled thereto. The follower 116 rides on a rotating cam 118. The follower assembly 114, follower 116, cam 118, and diaphragm 120 make up a triggering mechanism 122. The nasal drug delivery system 100 of FIG. 1 is shown essentially in the stored or unarmed position. FIG. 2 depicts the nasal drug delivery system 100 of FIG. 1, in the armed position. To move from the stored position of FIG. 1 to the armed position of FIG. 2 the cover 104 is rotated uncovering the mouthpiece 106 and the nosepiece 108. When in the armed position as shown in FIG. 2, a user may blow into the mouthpiece 106, which will cause the diaphragm 120 to expand. The cam 118 which in one embodiment is affixed to the diaphragm 120 rotates. The rotation of the cam 118 causes the follower 116 to slide off of the cam 118. The sliding of the follower 116 off of the cam 118 allows the follower assembly 114 to rotate. The rotation of the follower assembly 114 allows the spring 112, which is acting on the drug storage container 110 to force the drug storage container from a armed position as shown in FIG. 3 to a discharge position as shown in FIG. 4. One of skill in the art will appreciate that a drug storage container 110, such as a MDI works to allow the release of a predetermined amount of medicament upon the depression of the valve assembly 124. Accordingly no more than a predetermined amount of the medicament is released by each actuation of the nasal drug delivery system 100. The discharged medicament travels along the nosepiece 108, which has a lumen formed there through and is administered to the nose of the user. It has been observed that when a human expels air from the lungs though only the mouth, and particularly in instances where the exhalation is impeded by something that creates a backpressure in the pulmonary system, the soft palate operates to isolate and insulate the nasal pharynx from the remainder of the pulmonary system. That is the soft palate acts as a natural check valve preventing the flow of air between the lungs and the nasal cavity. Accordingly, by utilizing the backpressure created by the diaphragm 120 when a user is blowing into the mouthpiece 106, the one aspect of the instant invention is that it is capable of sufficiently isolating the lungs from the nasal cavity in conformity with the FDA Guidelines.

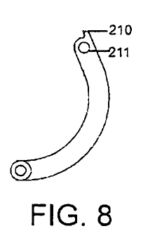

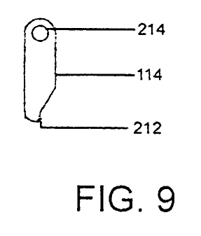

FIGS. 8 and 9 are close-up views of the cam 118 and the follower 116. As can be seen the cam 118 includes a lip 210. As described above, the cam when acted upon by the diaphragm 120 rotates. In one embodiment the cam 118 rotates about a pin (not shown) inserted into the lumen 211. The lip 210, when rotating acts on the follower 116, which as shown in FIG. 9 has a corresponding lip 212, which rests on the cam 118, and is acted upon by the lip 210. The movement of the diaphragm 120 and cam 118 causes the follower 116 to be forced off of the cam 118 by the lip 210 acting on the lip 212 of the follower. In one embodiment the follower 114 rotates on a pin 214, which connects the follower 116 to the follower assembly 114. The rotation of the follower 116 allows the follower assembly to rotate due to the force applied by the spring 112, and causes the nasal drug delivery system 100 to dispense medication.

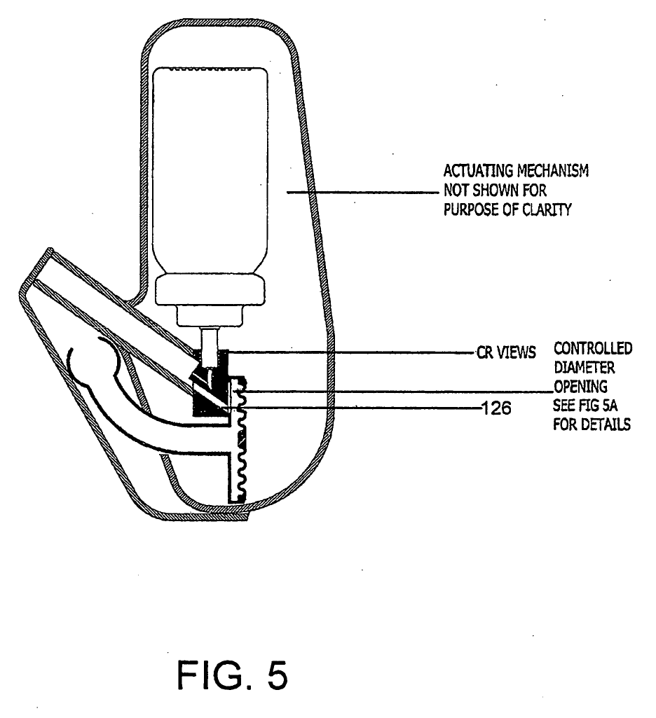

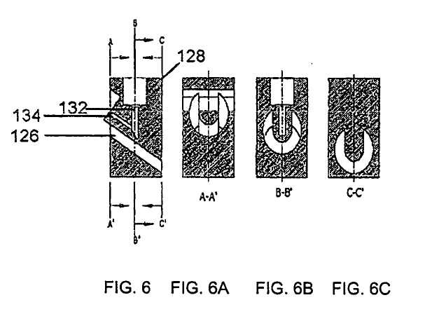

As described above, the mouthpiece 106 and the nosepiece 108 may in one embodiment of the present invention be in fluid communication with one another by a connecting lumen 126. This may be seen more clearly by reference to FIG. 5. As shown in FIG. 5, the connecting lumen allows for a portion of the air pressure forced into the mouthpiece 106 and against the diaphragm 120 to be bled off as a relatively high-pressure airflow. This high-pressure airflow helps to create turbulent flow of the medicament and direct the medicament in the direction of the user's nose as it is discharged from the drug storage container 110. In this way the user is assured that the majority of the medicament that is dispensed to the nosepiece 108 reaches the nasal passages to provide effective dosing. Naturally, one of skill in the art will appreciate that the diameter of the connecting lumen 126 must be kept relatively small to ensure that sufficient back pressure is created to ensure closure of the user's nasal pharynx and the prevention of medication from reaching the user's lungs. FIG. 6 is a close up view of the connecting lumen 126 and the medication dispensing bock 128. As shown in FIG. 6, the valve assembly 124 of the drug storage container 110 mates with a valve receiver 130. In one embodiment of the present invention, when the spring 112 acts on the drug storage container 110, the valve assembly 124 is kept vertically in place in relation to the dispensing block 128. The drug storage container 110 is moved vertically in relation to the valve assembly 124, allowing the release of a predetermined amount of medicament. The medicament is directed first into a vertical lumen 132, and then into an angled lumen 134. The angled lumen is such that it is substantially in alignment with the nosepiece 108.

The medicament in the drug storage container 110 is in at least one embodiment under pressure greater than atmospheric pressure. As shown in FIG. 6, the diameter of the valve receiver 130, is substantially greater than that of the vertical lumen 132, which in turn is greater than that of the angled lumen 134. Due to the change in diameter and the pressure of the medicament as it exits the drug storage device 110, the medicament enters the lumen having a relatively high velocity, and as a fine mist. As discussed above, the smaller the particles, the better the absorption by the nasal tissues. The connecting lumen 126 provides greater air volume flow to assist in the transfer of the fine mist of high velocity medicament into the nasal passages for absorption. As can be seen from FIGS. 6A-C, which are cross sections views of the dispensing block 128 cut along lines A-A′-C-C′ respectively, the connecting lumen may not be a lumen at all but rather a U-shaped passage formed in the dispensing block. As shown in FIGS. 6A-6C, the volume of air capable of passage through the U-shaped passage is far in excess of that through the angled lumen 134, thus providing ample volume ensuring complete dispensation of the medicament from the nosepiece 108 and into the nasal passages of the user.

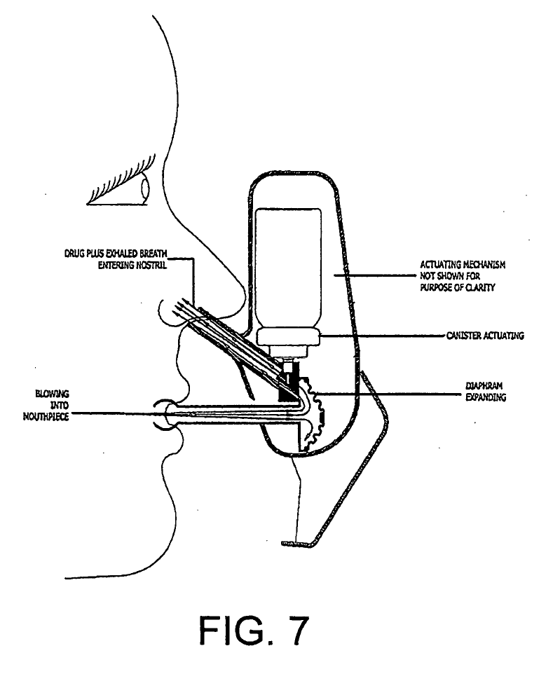

The combined actions of the release of medicament from the drug storage container 110, the breathing in by the user expanding the diaphragm 120, the flow of air through the connecting lumen and the mixing of the medicament and the airflow through the connecting lumen can be seen in FIG. 7.

Thus the nasal drug dispenser 100 described above has the ability to provide extremely small particles needed to coat the superior turbinates and epithelia yet prevent unwanted aerosolized droplets from entering the pulmonary system.

While the invention has been described in connection with what is considered to be the most practical and preferred embodiment, it should be understood that this invention is not limited to the disclosed embodiments, but on the contrary, is intended to cover various modifications and equivalent arrangements included within the spirit and scope of the appended claims.

What is claimed is:

1. A nasal drug delivery system comprising:
a container containing a substance to be delivered into the nasal passages of a user;
a nasal shaft extending from the drug delivery system for placement in or around the nose of user, said nasal shaft in fluid communication with a valve of said container;
an oral shaft, extending from the drug delivery system for placement in the mouth of a user; and
a pressure activated diaphragm trigger means, wherein upon application of pressure to the oral shaft the diaphragm trigger means releases a predetermined amount of said substance from said container to said nasal shaft.

2. The nasal drug delivery system of claim 1 wherein the oral shaft is in fluid communication with said nasal shaft.

3. The nasal drug delivery system of claim 2, wherein the pressure activated diaphragm trigger means comprises a follower.

4. The nasal drug delivery system of claim 3, wherein the pressure activated diaphragm trigger means comprises a follower assembly.

5. The nasal drug delivery system of claim 4, wherein the pressure activated diaphragm trigger means comprises a cam.

6. The nasal drug delivery system of claim 1, further comprising an activation spring.

7. A method of delivering a predetermined amount of medication to the nasal epithelia of a user comprising the steps of:
providing a drug applicator having a container containing a substance to be delivered, a nasal shaft, extending from the drug applicator for placement in or around the nose of user, with said nasal shaft in fluid communication with a valve of said container, an oral shaft extending from the drug applicator for placement in the mouth of a user, and a pressure activated diaphragm trigger means;
inserting the oral shaft into the mouth of a user;
inserting the nasal shaft into or around the nose of a user;
pressurizing the oral shaft which activates the diaphragm trigger means; and
releasing a predetermined amount of medicament from the container into the nasal shaft and into the nasal passage of the user.

8. The method of claim 7, wherein the drug applicator further includes a lumen connecting the nasal shaft and the oral shaft and further comprising a step of causing an airflow from said oral shaft to said nasal shaft.

9. The method of claim 8, wherein the airflow assists in dispersing the medication into the nasal passage of the user.

10. The method of claim 7, wherein pressurizing of the oral shaft displaces a diaphragm.

11. The method of claim 10, wherein the displacement of the diaphragm displaces a cam.

12. The method of claim 11, wherein displacement of the cam releases a follower and triggers release of the medicament.
